(19) United States
(12) Patent Application Publication
(10) Pub. No.: US 2023/0148605 A1
(43) Pub. Date: May 18, 2023

(54) APPLICATION OF GERANIOL IN PREPARATION OF FORMULATION FOR PROMOTING SYNTHESIS OF PSEUDOMONAS AERUGINOSA 3OC12-HSL SIGNAL MOLECULES

(71) Applicant: Institute of Microbiology, Guangdong Academy of Sciences (Guangdong Detection Center of Microbiology, Guangzhou (CN)

(72) Inventors: Xiaobao XIE, Guangzhou (CN); Wenru LI, Guangzhou (CN); Taohua ZENG, Guangzhou (CN); Zhiqing ZHANG, Guangzhou (CN); Qingshan SHI, Guangzhou (CN)

(73) Assignee: Institute of Microbiology, Guangdong Academy of Sciences (Guangdong Detection Center of Microbiology, Guangzhou (CN)

(21) Appl. No.: 17/915,527

(22) PCT Filed: Jan. 24, 2022
(86) PCT No.: PCT/CN2022/073419

§ 371 Date: Sep. 29, 2022

(30) Foreign Application Priority Data
Jun. 21, 2021 (CN) 202110685800.1

(51) Int. Cl. A01N 49/00; C12N 1/20; C12N 1/38

(52) U.S. Cl. CPC A01N 49/00; C12N 1/20; C12N 1/38

(57) ABSTRACT

An application of geraniol in preparation of formulation for promoting synthesis of Pseudomonas aeruginosa 3OC₁₂-HSL signal molecules is provided. It was found that geraniol slightly inhibits growth of Pseudomonas aeruginosa PAO1 strain, but can significantly promote synthesis of the 3OC₁₂-HSL signal molecules of the bacterium, and thus can be applied to preparation of formulation for promoting synthesis of the Pseudomonas aeruginosa 3OC₁₂-HSL signal molecules.

### CROSS REFERENCE TO THE RELATED APPLICATIONS

This application is the national phase entry of International Application No. PCT/CN2022/073419, filed on Jan. 24, 2022, which is based upon and claims priority to Chinese Patent Application No. 202110685800.1, filed on Jun. 21, 2021, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBKY080_Sequence_Listing.txt, created on 09/05/2022, and is 6,095 bytes in size.

### TECHNICAL FIELD

The present invention belongs to the field of harmful microorganism prevention and control technologies, and more particularly, relates to application of geraniol in preparation of formulation for promoting synthesis of Pseudomonas aeruginosa 3OC₁₂-HSL signal molecules.

### BACKGROUND

Pseudomonas aeruginosa (PA) is an opportunistic pathogen that may easily cause infections in burnt sites, respiratory tracts, and urinary tracts, which has little effect on healthy people, but may endanger lives of immunocompromised patients and those with cystic fibrosis. PA is highly resistant to most antibiotics, and may develop drug resistance through gene mutation or acquisition of exogenous resistant genes, or transient changes in gene and protein expression due to environmental stimuli, resulting in adaptive resistance. Pseudomonas aeruginosa always has a high infection rate and is hard to eradicate; in addition, extensive use of antibiotics has led to spread of multidrug-resistant Pseudomonas aeruginosa, making a task of preventing and controlling Pseudomonas aeruginosa more arduous, so it is urgent to find a new prevention and control strategy, and inhibition of quorum sensing (QS) of Pseudomonas aeruginosa has become a research hotspot. Quorum sensing is a density-dependent communication mechanism between bacterial individuals; and production of virulent factors such as biofilm, pyocyanin, and elastase, etc. of Pseudomonas aeruginosa are all regulated by the quorum sensing system of the bacterium. Inhibition of quorum sensing may inhibit virulence and pathogenicity of the bacterium, but has little influence on growth thereof, and thus fails to provide a favorable selection pressure for drug-resistant bacteria. Therefore, research and development of quorum sensing inhibitors is of great significance to solve the problem of bacterial drug resistance.

There are three quorum sensing systems of Pseudomonas aeruginosa, namely, las, rhl and pqs systems. The bacterium has the las system composed of lasI and lasR, the rhl system composed of rhlI and rhlR, and the pqs system composed of pqsABCDE, pqsH and pqsR, wherein, lasI, rhlI, pqsABCDE and pqsH are encoding genes of signal molecule synthase, respectively encoding 3OC₁₂-HSL, C₄-HSL, HHQ and PQS signal molecules; and lasR, rhlR and pqsR are encoding genes of corresponding signal molecule receptor proteins. The three quorum sensing systems, las, rhl and pqs, jointly regulate production of many virulent factors of Pseudomonas aeruginosa, including extracellular protease LasA, elastase LasB, pyocyanin and biofilm, etc.

Geraniol is an acyclic monoterpenoid, and is one of main components of rose oil and citronella oil. Geraniol is a colorless to yellow oily liquid at room temperature with a mild rose fragrance, and is usually used to make daily essence and edible essence.

### SUMMARY

An objective of the present invention is to provide application of geraniol in preparation of formulation for promoting synthesis of Pseudomonas aeruginosa 3OC₁₂-HSL signal molecules.

The present invention shows through experiments that geraniol may slightly inhibit growth of PAO1 [(Pseudomonas aeruginosa) PAO1] strain, but can significantly promote synthesis of 3OC₁₂-HSL signal molecules of the bacterium.

Therefore, the objective of the present invention is to provide application of geraniol in preparation of formulation for promoting synthesis of Pseudomonas aeruginosa 3OC₁₂-HSL signal molecules.

The Pseudomonas aeruginosa is preferably Pseudomonas aeruginosa PAO1.

The 3OC₁₂-HSL signal molecule is a signal molecule of the las quorum sensing system of the bacterium, and is synthesized by signal molecule synthase LasI.

Concentration of geraniol in a Pseudomonas aeruginosa culture solution is 0.313 μL/mL to 2.5 μL/mL.

It is found in the present invention that, geraniol with the concentration of 0.313 μL/mL to 2.5 μL/mL slightly inhibits growth of Pseudomonas aeruginosa PAO1 strain, but can significantly promote synthesis of the 3OC₁₂-HSL signal molecules of the bacterium, and thus may be applied to preparation of formulation for promoting synthesis of the Pseudomonas aeruginosa 3OC₁₂-HSL signal molecules.

### BRIEF DESCRIPTION OF THE DRAWINGS

FIG. 1 is a growth curve of Pseudomonas aeruginosa PAO1 under an action of geraniol.

FIG. 2 is influence of geraniol on expression of key genes in the Pseudomonas aeruginosa quorum sensing system and virulent genes regulated thereby.

### DETAILED DESCRIPTION

The following embodiments are further illustrations of the present invention, rather than limitations to the present invention.

### Embodiment 1

Preparation of PAO1 [(Pseudomonas aeruginosa) PAO1] bacterial suspension: a PAO1 culture medium in an exponential growth phase was sampled, centrifuged, washed once with PBS buffer, resuspended in PBS, and had a bacterial concentration diluted to 10⁸ CFU/mL to obtain PAO1 bacterial suspension.

1. Experiment on Influence of Geraniol on Pseudomonas aeruginosa PAO1 Growth

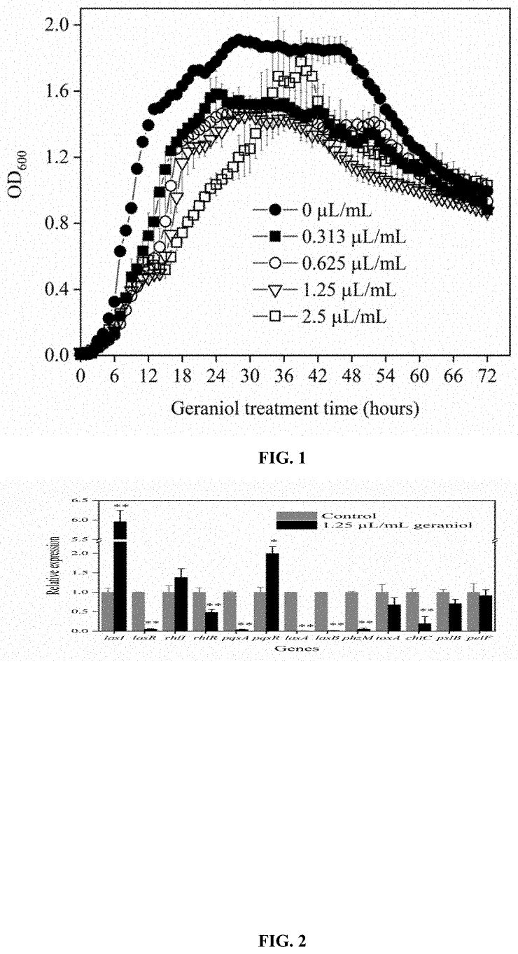

An LB medium and geraniol were respectively added to test tubes, joined with the PAO1 bacterial suspension in the exponential growth phase, to make total volumes all reach 10 mL, so that PAO1 bacterial concentrations were all 10⁶ CFU/mL, and concentrations of geraniol were respectively 0 μL/mL (control), 0.313 μL/mL, 0.625 μL/mL, 1.25 μL/mL, and 2.5 μL/mL. Samples from the experimental groups were respectively added to a honeycomb culture plate dedicated to an automatic growth curve analyzer (Bioscreen C), which has each well added with 350 μL culture medium; and each experimental group has three parallel experiments. The honeycomb culture plate was placed in the automatic growth curve analyzer, for shaking culture at 37° C. for 3 days, with OD₆₀₀ measured every hour. Taking OD₆₀₀ as an ordinate and culture time as an abscissa, a growth curve of PAO1 under an action of geraniol was drawn to study influence of geraniol on PAO1 growth. Results are shown in FIG. 1.

2. Experiment on Influence of Geraniol on Expression of Key Genes in the Pseudomonas aeruginosa Quorum Sensing System and Virulent Genes Regulated Thereby.

The PAO1 bacterial suspension in a logarithmic growth phase was added to 50 mL sterile LB liquid medium to make a final concentration of the bacterial solution reach 10⁶ CFU/mL. Geraniol was added to make a final concentration become 0 μL/mL (three biological replicates in a control group were respectively named A1, A2 and A3) and 1.25 μL/mL (three biological replicates in an experimental group were respectively named B1, B2 and B3); the respective groups were cultured at 37° C. and 180 rpm for 5 h, centrifuged to collect Bacteria, and then snap-frozen at −80° C. for later use.

Total bacterial RNA was extracted with a kit of Trizol (Thermo Company). After extraction, purity of RNA was detected with an ultra-micro spectrophotometer (Implen, Munich, Germany). An A260/A280 value of each RNA sample should be between 1.8 and 2.0. Reverse transcription and real-time fluorescent quantitative PCR amplification were performed with the PrimeScript RT Master Mix kit (Takara, Dalian, China) and the ETC 811 PCR instrument (Beijing Eastwin Life Sciences, Inc.). A q-PCR reaction system adopted Takara's SYBR Premix Ex TaqII (Tli RNaseH Plus) (Code No. RR820A); the PCR program was pre-denaturation at 95° C. for 30 s; denaturation at 95° C. for 5 s, annealing at 60° C. for 34 s, 40 loops; according to the 10 gene sequences already published on the GenBank website, primers for q-PCR were designed with Primer Premier 5.0 software, and meanwhile, the 16SrRNA gene was used as an internal reference gene. Primer sequence parameters are shown in Table 1.

Table 1 Genes and primer sequences thereof used in real-time fluorescent quantitative PCR

TABLE 1
Genes and primer sequences thereof used in real-time fluorescent quantitative PCR

| | | | | SEQ ID |
|---|---|---|---|---|
| Gene name | Gene locus | Gene description | Primer sequence (5′-3′) | NO: |
| 16S rRNA | PA5369.5 | 16S ribosomal RNA | GCGCAACCCTTGTCCTTAGTT (F) | 1 |
| | | | TGTCACCGGCAGTCTCCTTAG(R) | 2 |
| lasI | PA1432 | Acyl-homoserine-lactone | TGCGTGCTCAAGTGTTCAAGG (F) | 3 |
| | | synthase | CGGCTGAGTTCCCAGATGTGC(R) | 4 |
| lasR | PA1430 | ptional regulator LasR | GACCAGTTGGGAGATATCGGTTA (F) | 5 |
| | | | TCCGCCGAATATTTCCCATA (R) | 6 |
| rhlI | PA3476 | Acyl-homoserine-lactone | AAACCCGCTACATCGTCGC (F) | 7 |
| | | synthase | TCTCGCCCTTGACCTTCTGC (R) | 8 |
| rhLR | PA3477 | Transcriptional regulator | ATCGCCATCATCCTGAGCATT (F) | 9 |
| | | RhlR | TCGGAGGACATACCAGCACAC (R) | 10 |
| pqsA | PA0996 | Anthranilate-CoA ligase | GCAATACACCTCGGGTTCCA (F) | 11 |
| | | | TCCGCTGAACCAGGGAAAGA (R) | 12 |
| pqsR | PA1003 | Transcriptional regulator | TCGTTCTGCGATACGGTGAG (F) | 13 |
| (mvfR) | | MvfR | GCACTGGTTGAAGCGGGAG(R) | 14 |
| lasA | PA1871 | Protease LasA | GCCGCTGAATGACGACCTGT (F) | 15 |
| | | | TCAGGGTCAGCAACACTT (R) | 16 |
| lasB | PA3724 | Elastase LasB | AAGGCCTTGCGGGTATCC (F) | 17 |
| phzM | PA4209 | Phenazine-specific | GAATGGAAGTCCCGTTGC (F) | 19 |
| | | methyltransferase | GCCCTCGACATCCCTCA (R) | 20 |
| chiC | PA2300 | Chitinase | CTGGGAGTTCCGCAAGCGTTAC (F) | 21 |
| | | | ATCGGTGGCGGTGACGAAATAG (R) | 22 |
| toxA | PA1148 | Exotoxin A | CCCGGCGAAGCATGAC (F) | 23 |
| | | | GGGAAATGCAGGCGATGA(R) | 24 |
| pslB | PA2232 | Biofilm formation protein | CAACGAATCCACCTTCATCC(F) | 25 |
| | | PslB | ACTCGCCGCTCTGTACCTC(R) | 26 |
| pelF | PA3059 | Pellicle/biofilm biosynthesis | GACTTTCTCCACAGCAAG (F) | 27 |
| | | glycosyltransferase PelF | CAGAAGTAATTGACGAAGGA (R) | 28 |

### EXPERIMENTAL RESULTS

The results of growth curves of Pseudomonas aeruginosa PAO1 under action of different concentrations of geraniol are shown in FIG. 1. The experimental results show that geraniol has a concentration-dependent inhibitory effect on growth of PAO1 cells; and the higher the concentration of geraniol, the stronger the antibacterial effect. The 0.313 μL/mL, 0.625 μL/mL, 1.25 μL/mL and 2.5 μL/mL geraniol all slightly inhibit growth of PAO1, especially, the 2.5 μL/mL geraniol treatment group has the highest antibacterial activity. The 2.5 μL/mL geraniol treatment group has a bacterial proliferation rate in the logarithmic growth phase significantly lower than other groups, but has a longer logarithmic growth phase than other groups, and a very short stationary phase, the bacteria have been in slow proliferation after a lag phase, and then enter a decline phase; as a result, a bacterial concentration in the 2.5 μL/mL geraniol treatment group during a period of 33 h to 41 h is even higher than that in other geraniol treatment groups. In conclusion, the 0.313 μL/mL, 0.625 μL/mL, 1.25 μL/mL and 2.5 μL/mL geraniol all slightly inhibit growth of PAO1 cells.

After treating PAO1 cells with 1.25 μL/mL geraniol for 5 h, transcription levels of key genes of the quorum sensing system and related virulent genes are as shown in FIG. 2. Expression of the signal molecule synthase encoding gene lasI in the las system is up-regulated, and expression of the signal molecule receptor protein encoding gene lasR is down-regulated. Expression of the signal molecule receptor protein encoding gene rhlR in the rhl system is down-regulated, and an expression level of the signal molecule synthase encoding gene rhl does not change significantly. In the pqs system, expression of the signal molecule synthase encoding gene pqsA is down-regulated, and expression of the signal molecule receptor protein encoding gene pqsR is up-regulated. Expression levels of virulent genes lasA, lasB, phzM and chiC are significantly down-regulated, while expression levels of toxA, pslB and pelF are not significantly down-regulated. Thus, it can be seen that, geraniol can inhibit expression of the signal molecule receptor protein encoding gene of the Pseudomonas aeruginosa quorum sensing system las and rhl, and inhibit expression of the signal molecule synthase encoding gene of the pqs system, which further inhibits regulatory pathways of the three quorum sensing systems, and inhibits production of virulent factors regulated by the three quorum sensing systems, thereby controlling virulence and pathogenicity of Pseudomonas aeruginosa.

To sum up, the experimental results show that low concentration of geraniol slightly inhibits growth of Pseudomonas aeruginosa PAO1 strain (FIG. 1), but can significantly promote synthesis of 3OC₁₂-HSL signal molecules of the bacterium.

What is claimed is:

1. An application of geraniol in a preparation of a formulation for promoting a synthesis of Pseudomonas aeruginosa 3OC₁₂-HSL signal molecules.

2. The application according to claim 1, wherein Pseudomonas aeruginosa is Pseudomonas aeruginosa PAO1.

3. The application according to claim 1, wherein each of the Pseudomonas aeruginosa 3OC₁₂-HSL signal molecules is a signal molecule of a las quorum sensing system of a bacterium, and is synthesized by a signal molecule synthase LasI.

4. The application according to claim 1, wherein a concentration of the geraniol in a Pseudomonas aeruginosa culture solution is 0.313 μL/mL to 2.5 μL/mL.

5. The application according to claim 2, wherein a concentration of the geraniol in a Pseudomonas aeruginosa culture solution is 0.313 μL/mL to 2.5 μL/mL.

6. The application according to claim 3, wherein a concentration of the geraniol in a Pseudomonas aeruginosa culture solution is 0.313 μL/mL to 2.5 μL/mL.


SEQUENCE LISTING (28 sequences)

SEQ ID NO: 1 - DNA, 21 bp, Artificial Sequence
  The sequence is synthetized.
gcgcaacccttgtccttagtt

SEQ ID NO: 2 - DNA, 21 bp, Artificial Sequence
  The sequence is synthetized.
tgtcaccggcagtctccttag

SEQ ID NO: 3 - DNA, 21 bp, Artificial Sequence
  The sequence is synthetized.
tgcgtgctcaagtgttcaagg

SEQ ID NO: 4 - DNA, 21 bp, Artificial Sequence
  The sequence is synthetized.
cggctgagttcccagatgtgc

SEQ ID NO: 5 - DNA, 23 bp, Artificial Sequence
  The sequence is synthetized.
gaccagttgggagatatcggtta

SEQ ID NO: 6 - DNA, 20 bp, Artificial Sequence
  The sequence is synthetized.
tccgccgaatatttcccata

SEQ ID NO: 7 - DNA, 19 bp, Artificial Sequence
  The sequence is synthetized.
aaacccgctacatcgtcgc

SEQ ID NO: 8 - DNA, 20 bp, Artificial Sequence
  The sequence is synthetized.
tctcgcccttgaccttctgc

SEQ ID NO: 9 - DNA, 21 bp, Artificial Sequence
  The sequence is synthetized.
atcgccatcatcctgagcatt

SEQ ID NO: 10 - DNA, 21 bp, Artificial Sequence
  The sequence is synthetized.
tcggaggacataccagcacac

SEQ ID NO: 11 - DNA, 20 bp, Artificial Sequence
  The sequence is synthetized.
gcaatacacctcgggttcca

SEQ ID NO: 12 - DNA, 20 bp, Artificial Sequence
  The sequence is synthetized.
tccgctgaaccagggaaaga

SEQ ID NO: 13 - DNA, 20 bp, Artificial Sequence
  The sequence is synthetized.
tcgttctgcgatacggtgag

SEQ ID NO: 14 - DNA, 19 bp, Artificial Sequence
  The sequence is synthetized.
gcactggttgaagcgggag

SEQ ID NO: 15 - DNA, 20 bp, Artificial Sequence
  The sequence is synthetized.
gccgctgaatgacgacctgt

SEQ ID NO: 16 - DNA, 18 bp, Artificial Sequence
  The sequence is synthetized.
tcagggtcagcaacactt

SEQ ID NO: 17 - DNA, 18 bp, Artificial Sequence
  The sequence is synthetized.
aaggccttgcgggtatcc

SEQ ID NO: 18 - DNA, 20 bp, Artificial Sequence
  The sequence is synthetized.
cgtgtacaaccgtgcgttct

SEQ ID NO: 19 - DNA, 18 bp, Artificial Sequence
  The sequence is synthetized.
gaatggaagtcccgttgc

SEQ ID NO: 20 - DNA, 17 bp, Artificial Sequence
  The sequence is synthetized.
gccctcgacatccctca

SEQ ID NO: 21 - DNA, 22 bp, Artificial Sequence
  The sequence is synthetized.
ctgggagttccgcaagcgttac

SEQ ID NO: 22 - DNA, 22 bp, Artificial Sequence
  The sequence is synthetized.
atcggtggcggtgacgaaatag

SEQ ID NO: 23 - DNA, 16 bp, Artificial Sequence
  The sequence is synthetized.
cccggcgaagcatgac

SEQ ID NO: 24 - DNA, 18 bp, Artificial Sequence
  The sequence is synthetized.
gggaaatgcaggcgatga

SEQ ID NO: 25 - DNA, 20 bp, Artificial Sequence
  The sequence is synthetized.
caacgaatccaccttcatcc

SEQ ID NO: 26 - DNA, 19 bp, Artificial Sequence
  The sequence is synthetized.
actcgccgctctgtacctc

SEQ ID NO: 27 - DNA, 18 bp, Artificial Sequence
  The sequence is synthetized.
gactttctccacagcaag

SEQ ID NO: 28 - DNA, 20 bp, Artificial Sequence
  The sequence is synthetized.
cagaagtaattgacgaagga